(19) United States
(12) Patent Application Publication
(10) Pub. No.: US 2024/0398256 A1
(43) Pub. Date: Dec. 5, 2024

(54) BODY PART DIMENSION MEASURING APPAREL AND METHOD OF USE

(71) Applicant: Jonathan Patrick Whitford, Surrey (CA)

(72) Inventor: Jonathan Patrick Whitford, Surrey (CA)

(21) Appl. No.: 18/325,236

(22) Filed: May 30, 2023

(51) Int. Cl. A61B 5/107; A61B 5/00; G01S 5/00; G01S 5/02

(52) U.S. Cl. CPC A61B 5/107; A61B 5/6804; G01S 5/0072; G01S 5/0284

(57) ABSTRACT

A body dimension measuring apparel that is provided in a plurality of styles wherein the present invention provides measurement and tracking of various body part dimensions such as but not limited to the waist. The present invention includes clothing that is provided in various styles wherein the clothing includes integrated thereinto a multitude of distance sensors. The distance sensors are arranged on the clothing of the present invention so as to circumferentially surround a body part such as but not limited to the waist. The distance sensors are operably coupled to each other and are further operably coupled to a controller. The controller is communicably coupled to a smartphone wherein the smartphone has loaded thereon a software application configured to provide operation of the present invention. The software application provides recording, storage and display of the body part dimensions received from the controller.

### FIELD OF THE INVENTION

The present invention relates generally to apparel, more specifically but not by way of limitation, apparel that is configured to provide automated measuring reporting and tracking of certain areas of the wearer of the apparel.

### BACKGROUND

Personal fitness is a significant industry and millions of individuals engage in fitness activities as part of a healthy lifestyle. Diet and exercise are the two most important factors when managing an individual's fitness. Healthy foods such as but not limited to vegetables and lean proteins assist an individual in maintaining a proper weight and can further assist an individual in losing weight. Exercise regimens are typically combined with a diet when attempting to attain a fitness goal such as but not limited to weight loss.

While individuals are pursuing fitness goals it is common for the individual to measure parameters such as but not limited to weight and body area dimensions. The aforementioned parameters are measured and utilized to ensure progress is being made and can further serve as a motivator for the individual. Weight is typically measured by a weight scale and is utilized on a routine basis in order to track the progress of an individual. Many individuals are further working on certain parts of their body such as but not limited to their waist or thighs. The individuals have an objective to change the dimension/size of these areas and will attempt to measure their progress as they engage in their fitness routine. Devices such as measuring tape is utilized but this is an inaccurate measurement and can sometimes require assistance. Body scanning devices are further employed but these types of devices are typically only available at gyms and can cost money for each use thereof.

Accordingly, there is a need for apparel that is configured to provide measurements of certain body areas wherein the apparel of the present invention is further communicably coupled to a smart phone wherein a software application of the present invention provides recording and tracking of the body dimensions.

### SUMMARY OF THE INVENTION

It is the object of the present invention to provide apparel that is configured to provide measurement of body part dimensions ensuing being donned by a user wherein the present invention includes a plurality of distance sensors located through the apparel.

Another object of the present invention is to provide a method for monitoring dimensions of body parts with apparel wherein the plurality of distance sensors are communicably coupled and circumferentially disposed around certain body parts ensuing the apparel being donned by a wearer.

A further object of the present invention is to provide apparel that is configured to provide measurement of body part dimensions ensuing being donned by a user wherein the present invention further includes a controller integrated into the apparel.

Yet a further object of the present invention is to provide a method for monitoring dimensions of body parts with apparel wherein the controller is communicably coupled to a smart phone.

Still another object of the present invention is to provide apparel that is configured to provide measurement of body part dimensions wherein the present invention further includes a software application configured to receive, store and report dimensions for body parts identified by a wearer of the apparel of the present invention.

An additional object of the present invention is to provide a method for monitoring dimensions of body parts with apparel wherein the apparel of the present invention has distance sensors around areas of the body such as but not limited to the waist, legs and buttocks.

Yet a further object of the present invention is to provide apparel that is configured to provide measurement of body part dimensions wherein the apparel of the present invention is provide in numerous alternate styles.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a perspective view of exemplary apparel of the present invention; and

FIG. 2 is a flow chart diagram of the method of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated body dimension measuring apparel 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

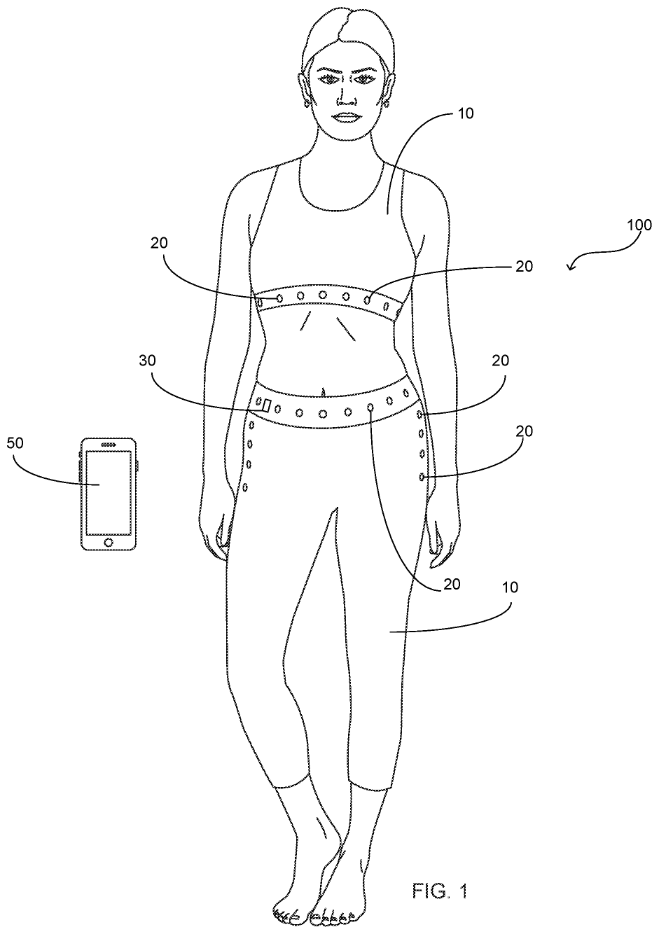

Referring in particular to the Figures submitted herewith, the body dimension measuring apparel 100 is illustrated herein in FIG. 1 in an exemplary style. It should be understood within the scope of the present invention that the style of apparel illustrated herein is exemplary only and that the present invention could be provided in numerous styles of apparel. It should be understood within the scope of the present invention that all styles of apparel of the present invention are fitted to the wearer such that the clothing layer 10 is adjacent to and against the skin of the wearer in order to achieve the desired objective of measuring the dimension of the body part. It is contemplated within the scope of the present invention that the clothing layer 10 is manufactured from materials such as but not limited to spandex, nylon, polyester or cotton.

The clothing layer 10 of the body dimension measuring apparel 100 includes a plurality of distance sensors 20 integrated thereinto. The distance sensors 20 are integrated into the clothing layer 10 utilizing suitable durable techniques such as but not limited to stitching. The distance sensors 20 are communicably coupled utilizing thin wire or other technique and are operable to transmit a signal therebetween. While various signal transmissions could be employed, in a preferred embodiment of the present invention the distance sensors 20 employ time of flight to determine the distance in between each of the distance sensors 20. As is known in the art, time of flight is configured to identify a distance between two sensors based on the time of travel for the signal from one sensor to another sensor. The distance sensors 20 are placed around various body parts in order to provide dimension measuring thereof. By way of example and as illustrated herein, the distance sensors 20 are circumferentially mounted around the waist area, upper torso area and along the outer thighs. It should be understood within the scope of the present invention that the distance sensors 20 could be placed in various alternate locations in order to attain measurements of different body parts.

The distance sensors 20 are communicably coupled to controller 30. Controller 30 has the necessary electronics to receive, store, manipulate and transmit data. The controller 30 functions to receive all of the data transmissions between the distance sensors 20 and subsequently transmit to smartphone 50. It should be understood within the scope of the present invention that the distance sensors 20 are assigned an identifier based on their location and are grouped in association with the body part to which the sensors are proximate. By way of example but not limitation, the distance sensors 20 circumferentially disposed around the waist area have an identifier such as W1, W2, etc., and the data transmitted therebetween is identified as being the data for the waist measurement of the wearer of the body dimension measuring apparel 100. The controller 30 is integrated into the clothing layer 10 utilizing suitable techniques.

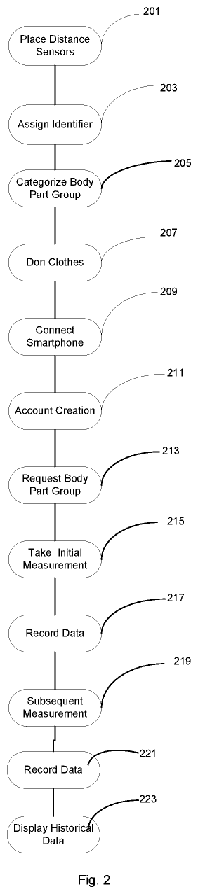

Referring in particular to FIG. 2 submitted herewith, a flowchart of the method of use of the body dimension measuring apparel 100 is diagrammed therein. In step 201, the distance sensors are placed in desired locations in the clothing layer 10 as previously discussed herein. Step 203, each of the distance sensors 20 is assigned an identifier wherein the identifier provides an indication of the location of the distance sensor 20 on the clothing layer 10. By way of example but not limitation, an identifier of B1 would indicate a first distance sensor on the buttocks area of the clothing layer. In step 205, the group of distance sensors 20 located on a particular body part are categorized into body part groups. For example, all of the distance sensors 20 circumferentially present around the waist area are associated and categorized into a waist group. In step 207, a wearer will don the clothes of the present invention. Step 209, the wearer will connect the clothes donned to a smartphone 50 wherein the smartphone 50 has thereon a software application configured to provide operation of the body dimension measuring apparel 100. In step 211, the wearer will create an account wherein the account will contain various demographic and target parameters for the wearer. Step 213, the software application will request to the wearer what body part or parts does the user desire to take measurements thereof. In step 215, the body dimension measuring apparel 100 will initiate the inaugural measurement of the requested body part or parts. Step 217, the initial measurement of the body part or parts are recorded and stored on the smartphone 50. In step 219, after an elapsed period of time, the wearer will don the clothes of the present invention again and initiate through the software application a request to make a subsequent measurement of the body part or parts having had the inaugural measurement recorded. Step 221, the subsequent measurement of the body part or parts are recorded. In step 223, the wearer employs the software application to display historical data for the body part or parts wherein the measurement data is displayed for comparison.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A body dimension measuring apparel that is configured to provide measurement of at least one body part wherein the body dimension measuring apparel comprises:
a clothing layer, said clothing layer having a plurality of distance sensors, said distance sensors being placed so as to be adjacent a body part, said distance sensors being operably coupled, said distance sensors configured to determine a distance between each sensor; and
a controller, said controller being integrally mounted in said clothing layer, said controller being operably coupled to said plurality of distance sensors, said controller having necessary electronics to receive, store, manipulate and transmit data, said controller being communicably coupled to a smartphone, said controller configured to transmit measurement data of the body part to the smartphone.

2. The body dimension measuring apparel that is configured to provide measurement of at least one body part as recited in claim 1, wherein the plurality of distance sensors are organized into body part groups, wherein the body part groups are adjacent independent body parts.

3. The body dimension measuring apparel that is configured to provide measurement of at least one body part as recited in claim 2, wherein a group of the plurality of distance sensors are circumferentially disposed about a waist of a wearer subsequent the wearer donning the body dimension measuring apparel.

4. The body dimension measuring apparel that is configured to provide measurement of at least one body part as recited in claim 3, wherein a group of the plurality of distance sensors are circumferentially disposed about a upper torso of a wearer subsequent the wearer donning the body dimension measuring apparel.

5. The body dimension measuring apparel that is configured to provide measurement of at least one body part as recited in claim 4, wherein a group of the plurality of distance sensors are circumferentially disposed about a buttocks area of a wearer subsequent the wearer donning the body dimension measuring apparel.

6. The body dimension measuring apparel that is configured to provide measurement of at least one body part as recited in claim 5, wherein a group of the plurality of distance sensors are circumferentially disposed about a thigh of a wearer subsequent the wearer donning the body dimension measuring apparel.

7. A method of measuring body part dimensions utilizing body dimension measuring apparel wherein the method comprises the steps of:
placing a plurality of distance sensors into a clothing layer of the body dimension measuring apparel;
assigning an identifier to each of the plurality of distance sensors;
categorizing the plurality of distance sensors into body part groups, wherein the body part groups represent a body part that a set of the plurality of distance sensors are adjacent or circumferentially present;
donning the body dimension measuring apparel, wherein a wearer will don the body dimension measuring apparel;
connecting the body dimension measuring apparel to a smartphone, wherein the body dimension measuring apparel includes a controller wherein the controller has necessary electronics to receive, store, manipulate and transmit data;
creating an account, wherein the wearer will create an account on a software application present on the smartphone, wherein the software application provides operation of the body dimension measuring apparel;
requesting a body part group, wherein the software group requests from the wearer what body part to take measurements thereof;
measuring the body part, wherein the distance sensors are employed to capture a measurement of the requested body part;
recording the measurement of the requested body part;
taking a subsequent measurement of the requested body part, wherein the wearer will initiate a subsequent measurement after an elapsed time period;
recording the subsequent measurement; and
displaying measurement data for the requested body part, wherein the software application provides historical measurement data to the wearer for the requested body part.
